(19) United States
(12) Patent Application Publication
(10) Pub. No.: US 2026/0294439 A1
(43) Pub. Date: Oct. 1, 2026

(54) LEFT ATRIAL APPENDAGE CLOSURE DEVICE

(71) Applicant: BOSTON SCIENTIFIC SCIMED, INC., MAPLE GROVE, MN (US)

(72) Inventors: Jan-Hung Chen, St. Paul, MN (US); Harishankar Natesan, Shoreview, MN (US); Thyna M. Chau, Woodbury, MN (US)

(73) Assignee: BOSTON SCIENTIFIC SCIMED, INC., MAPLE GROVE, MN (US)

(21) Appl. No.: 19/701,022

(22) Filed: Jun. 8, 2026

Related U.S. Application Data
(63) Continuation of application No. 18/490,986, filed on Oct. 20, 2023, now Pat. No. 12,653,539.
Application No. 18/490,986 is a continuation of application No. 17/857,513, filed on Jul. 5, 2022, now Pat. No. 12,383,278.
(60) Provisional application No. 63/219,696, filed on Jul. 8, 2021.

(51) Int. Cl. A61B 17/12

(52) U.S. Cl. CPC A61B 17/12172; A61B 17/12031; A61B 17/12122; A61B 2017/1205

(57) ABSTRACT

A left atrial appendage closure device may include an expandable framework having a plurality of struts joined at a proximal and distal hub. When the framework is fully constrained in a first position, a first segment of struts extends distally from the distal hub parallel to a central longitudinal axis to a first bend and a second segment of struts extends from the first bend proximally. A first amount of the framework is unconstrained in a second position, where the first segment extends distally from the distal hub parallel to the central longitudinal axis to the first bend, the second segment extends from the first bend proximally and radially outward to a second bend, a third segment of struts extends from the second bend proximally and radially inward to a third bend, and a fourth segment of struts extends from the third bend proximally to within the delivery sheath.

### CROSS-REFERENCE TO RELATED APPLICATIONS

This application is a continuation of U.S. application Ser. No. 18/490,986, filed Oct. 20, 2023, which is a continuation of U.S. application Ser. No. 17/857,513, filed Jul. 5, 2022, now U.S. Pat. No. 12,383,278, which claims the benefit of priority of U.S. Provisional Application No. 63/219,696 filed Jul. 8, 2021, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices and introducers as well as alternative methods for manufacturing and using medical devices and introducers.

### SUMMARY

In one example, a left atrial appendage closure device may comprise an expandable framework having a plurality of struts disposed about a central longitudinal axis, the plurality of struts being joined together at a proximal hub and a distal hub. When the expandable framework is fully constrained in a first position by a delivery sheath, a first segment of the plurality of struts extends distally from the distal hub parallel to the central longitudinal axis to a first bend and a second segment of the plurality of struts extends from the first bend proximally. A first amount of the expandable framework is unconstrained by the delivery sheath in a second position. In the second position the first segment of the plurality of struts extends distally from the distal hub parallel to the central longitudinal axis to the first bend, the second segment of the plurality of struts extends from the first bend proximally and radially outward to a second bend, a third segment of the plurality of struts extends from the second bend proximally and radially inward to a third bend, and a fourth segment of the plurality of struts extends from the third bend proximally to within the delivery sheath.

In addition or alternatively to any example disclosed herein, in the second position the first segment and the second segment form an acute angle opening inwardly toward an interior of the expandable framework.

In addition or alternatively to any example disclosed herein, a second amount of the expandable framework greater than the first amount is unconstrained by the delivery sheath in a third position. In the third position the first segment of the plurality of struts extends distally from the distal hub parallel to the central longitudinal axis to the first bend, the second segment of the plurality of struts extends from the first bend radially outward generally perpendicular to the central longitudinal axis to the second bend, the third segment of the plurality of struts extends from the second bend proximally and radially inward to the third bend, and the fourth segment of the plurality of struts extends from the third bend proximally toward the proximal hub disposed within the delivery sheath.

In addition or alternatively to any example disclosed herein, in the third position the second segment and the third segment form an acute angle opening inwardly toward the central longitudinal axis.

In addition or alternatively to any example disclosed herein, in the third position the third segment and the fourth segment form an obtuse angle opening outwardly away from the central longitudinal axis.

In addition or alternatively to any example disclosed herein, a third amount of the expandable framework greater than the second amount is unconstrained by the delivery sheath in a fourth position. In the fourth position the first segment of the plurality of struts extends distally from the distal hub parallel to the central longitudinal axis to the first bend, the second segment of the plurality of struts extends from the first bend distally and radially outward to the second bend, the third segment of the plurality of struts extends from the second bend proximally and radially inward toward the third bend, and the fourth segment of the plurality of struts extends from the third bend proximally toward the proximal hub disposed within the delivery sheath.

In addition or alternatively to any example disclosed herein, in the fourth position the first segment and the second segment form an obtuse angle opening inwardly toward an interior of the expandable framework.

In addition or alternatively to any example disclosed herein, in the fourth position the second segment and the third segment form an acute angle opening inwardly toward the central longitudinal axis.

In addition or alternatively to any example disclosed herein, in the fourth position the third segment and the fourth segment form an obtuse angle opening outwardly away from the central longitudinal axis.

In addition or alternatively to any example disclosed herein, a fourth amount of the expandable framework greater than the third amount is unconstrained by the delivery sheath in a fifth position. In the fifth position the first segment of the plurality of struts extends distally from the distal hub parallel to the central longitudinal axis to the first bend, the second segment of the plurality of struts extends from the first bend distally and radially outward to the second bend, the third segment of the plurality of struts extends from the second bend proximally to the third bend, and the fourth segment of the plurality of struts extends from the third bend radially inward toward the proximal hub.

In addition or alternatively to any example disclosed herein, in the fifth position the first segment and the second segment form an obtuse angle opening inwardly.

In addition or alternatively to any example disclosed herein, in the fifth position the second segment and the third segment form an acute angle opening inwardly.

In addition or alternatively to any example disclosed herein, in the fifth position the third segment and the fourth segment form an angle of about 90 degrees or less opening inwardly.

In addition or alternatively to any example disclosed herein, the distal hub is disposed proximal of the first bend.

In addition or alternatively to any example disclosed herein, in the second position the distal hub is disposed distal of the second bend.

In addition or alternatively to any example disclosed herein, a left atrial appendage closure device may comprise an expandable framework having a plurality of struts disposed about a central longitudinal axis, the plurality of struts being joined together at a proximal hub and a distal hub. As the expandable framework shifts from fully constrained to fully unconstrained, the expandable framework transitions sequentially through a plurality of positions. In a first position, a first segment of the plurality of struts extends distally from the distal hub parallel to the central longitudinal axis to a first bend and a second segment of the plurality of struts extends from the first bend proximally and generally parallel to the central longitudinal axis. In a second position, the second segment of the plurality of struts, if swept circumferentially around the central longitudinal axis, defines a generally conical shape tapering radially outward in a proximal direction from the first bend toward a second bend.

In addition or alternatively to any example disclosed herein, in the second position, the distal hub is disposed proximal of the first bend and the distal hub is disposed distal of the second bend.

In addition or alternatively to any example disclosed herein, in a third position, the second segment of the plurality of struts, if swept circumferentially around the central longitudinal axis, defines a generally planar shape oriented generally perpendicular to the central longitudinal axis.

In addition or alternatively to any example disclosed herein, in a fourth position, the second segment of the plurality of struts, if swept circumferentially around the central longitudinal axis, defines a generally conical shape tapering radially outward in a distal direction from the first bend toward the second bend.

In addition or alternatively to any example disclosed herein, a left atrial appendage closure device system may comprise a delivery sheath having a lumen extending therein, and a left atrial appendage closure device comprising an expandable framework having a plurality of struts disposed about a central longitudinal axis, the plurality of struts being joined together at a proximal hub and a distal hub. When the expandable framework is disposed within the lumen of the delivery sheath in a first position, a first segment of the plurality of struts extends distally from the distal hub parallel to the central longitudinal axis to a first bend and a second segment of the plurality of struts extends from the first bend proximally. Relative axial translation between the delivery sheath and the expandable framework exposes some of the expandable framework in a second position. In the second position the first segment of the plurality of struts extends distally from the distal hub parallel to the central longitudinal axis to the first bend, the second segment of the plurality of struts extends from the first bend proximally and radially outward to a second bend, a third segment of the plurality of struts extends from the second bend proximally and radially inward to a third bend, and a fourth segment of the plurality of struts extends from the third bend proximally to within the delivery sheath. Relative axial translation between the delivery sheath and the expandable framework exposes more of the expandable framework in a third position than in the second position. In the third position the first segment of the plurality of struts extends distally from the distal hub parallel to the central longitudinal axis to the first bend, the second segment of the plurality of struts extends from the first bend radially outward generally perpendicular to the central longitudinal axis to the second bend, the third segment of the plurality of struts extends from the second bend proximally and radially inward to the third bend, and the fourth segment of the plurality of struts extends from the third bend proximally toward the proximal hub disposed within the delivery sheath. Relative axial translation between the delivery sheath and the expandable framework exposes more of the expandable framework in a fourth position than in the third position. In the fourth position the first segment of the plurality of struts extends distally from the distal hub parallel to the central longitudinal axis to the first bend, the second segment of the plurality of struts extends from the first bend distally and radially outward to the second bend, the third segment of the plurality of struts extends from the second bend proximally and radially inward toward the third bend, and the fourth segment of the plurality of struts extends from the third bend proximally toward the proximal hub disposed within the delivery sheath. Relative axial translation between the delivery sheath and the expandable framework exposes all of the expandable framework in a fifth position. In the fifth position the first segment of the plurality of struts extends distally from the distal hub parallel to the central longitudinal axis to the first bend, the second segment of the plurality of struts extends from the first bend distally and radially outward to the second bend, the third segment of the plurality of struts extends from the second bend proximally to the third bend, and the fourth segment of the plurality of struts extends from the third bend radially inward toward the proximal hub.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-2 are side views of a left atrial appendage closure device system;

FIG. 3 illustrates selected aspects of a left atrial appendage closure device;

FIG. 4 illustrates selected aspects of the left atrial appendage closure device of FIG. 3; and

FIGS. 5-9 are schematic partial cross-sectional views illustrating selected aspects related to deploying the left atrial appendage closure device of FIGS. 3-4.

While aspects of the disclosure are amenable to various modifications and alternative forms, examples are shown in the drawings and described herein. It should be understood, however, that the intention is not to limit aspects of the disclosure to the particular embodiments described. On the contrary, the disclosure shall cover all modifications, equivalents, and alternatives falling within the spirit and scope thereof.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate exemplary aspects of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, all elements of the present disclosure are not necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The left atrial appendage may be attached to and in fluid communication with a left atrium of a patient's heart. In some patients, the left atrial appendage may have a complex geometry and/or irregular surface area. Those of skill in the art will also recognize that the medical devices and methods disclosed herein may be adapted for various sizes and shapes of the left atrial appendage, as necessary. The left atrial appendage may include a generally longitudinal axis arranged along a depth of a main body of the left atrial appendage. The main body may include a wall and an ostium forming a proximal mouth. In some embodiments, a lateral extent of the ostium and/or the wall may be smaller or less than a depth of the main body along the longitudinal axis, or a depth of the main body may be greater than a lateral extent of the ostium and/or the wall. In some embodiments, the left atrial appendage may include a tail-like element associated with a distal portion of the main body, which element may protrude radially or laterally away from the main body.

The following figures illustrate selected components and/or arrangements of a left atrial appendage closure device, a left atrial appendage closure device system, and/or methods of using the left atrial appendage closure device and/or the left atrial appendage closure device system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the left atrial appendage closure device and/or the left atrial appendage closure device system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

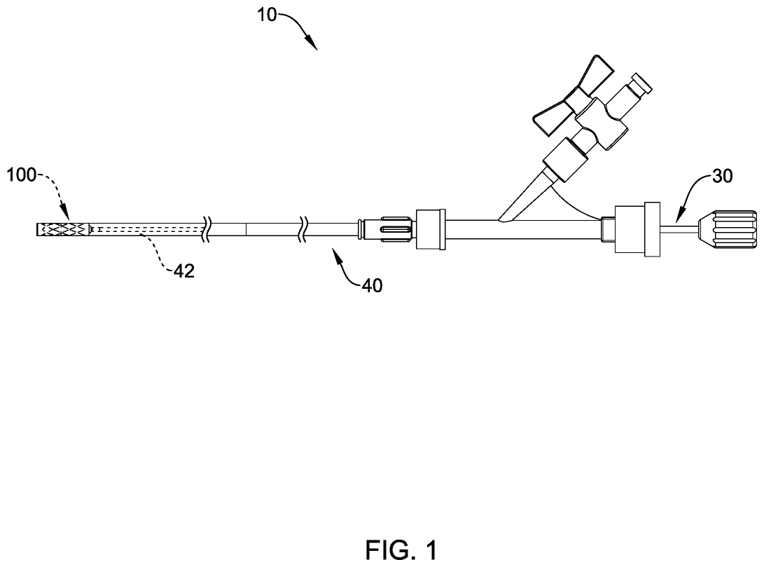

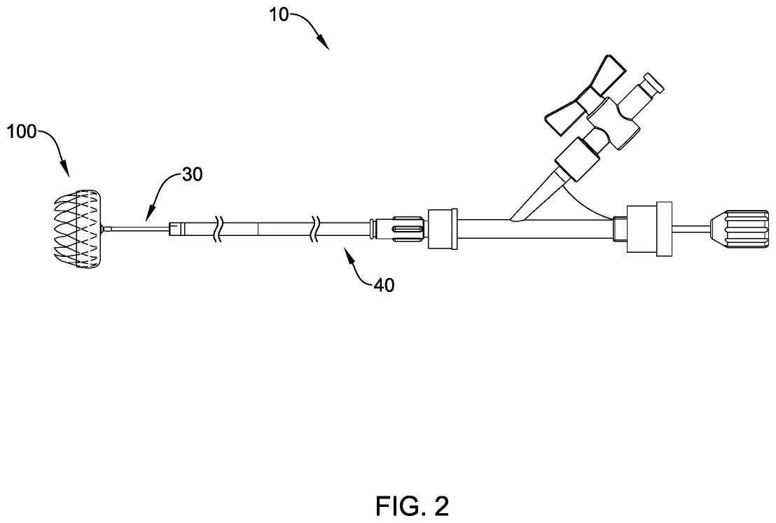

FIGS. 1-2 illustrate selected components and/or arrangements of a left atrial appendage closure device system 10 which may be used for occluding a left atrial appendage. It should be noted that in any given figure, some features of the left atrial appendage closure device system 10 may not be shown, or may be shown schematically, for simplicity. Additional details regarding some of the components of the left atrial appendage closure device system 10 may be illustrated in other figures in greater detail.

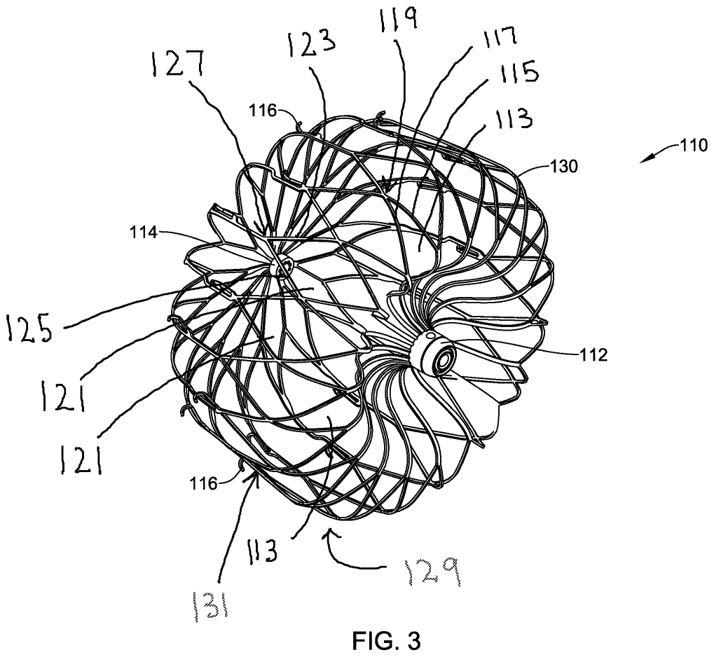

The left atrial appendage closure device system 10 may include a delivery sheath 40 having a lumen 42 extending from a proximal opening to a distal opening, a core wire 30 slidably disposed within the lumen 42, and a left atrial appendage closure device 100 for occluding the left atrial appendage. The left atrial appendage closure device 100 may include an expandable framework 110 (e.g., FIG. 3) configured to shift between a fully constrained configuration (e.g., FIG. 1), wherein the left atrial appendage closure device 100 is disposed within the lumen 42 proximate the distal opening in the delivery configuration, and a fully unconstrained configuration (e.g., FIG. 2), wherein the left atrial appendage closure device 100 and/or the expandable framework 110 is configured to shift between the fully constrained configuration and the fully unconstrained configuration as the left atrial appendage closure device 100 is translated relative to the delivery sheath 40. In at least some embodiments, the expandable framework 110 may be self-biased toward the fully unconstrained configuration.

The left atrial appendage closure device 100 may be disposed at and/or releasably securable to a distal portion of the core wire 30. The core wire 30 may be slidably and/or rotatably disposed within the lumen 42 of the delivery sheath 40. In some embodiments, a proximal end of the core wire 30 may extend proximally of a proximal end of the delivery sheath 40 and/or the proximal opening of the lumen 42 for manual manipulation by a clinician or practitioner. In some embodiments, the left atrial appendage closure device 100 may be removably attached, joined, secured, or otherwise connected to a distal end of the core wire 30. The core wire 30 may be configured to and/or may be capable of axially translating the left atrial appendage closure device 100 relative to the delivery sheath 40. In one example, the core wire 30 may be advanced distally while the delivery sheath 40 is held in a constant position. In another example, the core wire 30 may be advanced distally while the delivery sheath 40 is retracted proximally. In yet another example, the core wire 30 may be held in a constant position while the delivery sheath 40 is retracted proximally relative to the core wire 30 and/or the left atrial appendage closure device 100. Other configurations are also contemplated. The delivery sheath 40 and/or the core wire 30 may have a selected level of axial stiffness and/or pushability characteristics while also having a selected level of flexibility to permit navigation through the patient's vasculature.

Some suitable, but non-limiting, examples of materials for the left atrial appendage closure device system 10, the core wire 30, the delivery sheath 40, and/or the left atrial appendage closure device 100, etc. are discussed below. It is contemplated that any exemplary left atrial appendage closure device disclosed herein may be used in accordance with and/or be associated with the example left atrial appendage closure device system 10 described above.

The left atrial appendage closure device 100 may comprise an expandable framework 110 configured to shift axially and/or radially along a central longitudinal axis between the fully constrained configuration and the fully unconstrained configuration. In the fully constrained configuration, the expandable framework 110 may be axially elongated and/or radially compressed. In the fully unconstrained configuration, the expandable framework 110 may be axially shortened and/or radially expanded.

As seen in FIG. 3, which illustrates selected aspects of the left atrial appendage closure device 100 in the fully unconstrained configuration, the expandable framework 110 may have a plurality of struts disposed about the central longitudinal axis. In some embodiments, the plurality of struts may define a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements.

The expandable framework 110 may include a proximal hub 112 and a distal hub 114. In some embodiments, the proximal hub 112 and/or the distal hub 114 may be centered on and/or coaxial with the longitudinal axis. The plurality of struts may be joined together at and/or fixedly attached to the proximal hub 112 and/or the distal hub 114. The proximal hub 112 may be configured to releasably connect, secure, and/or attach the left atrial appendage closure device 100 and/or the expandable framework 110 to the core wire 30. In some embodiments, the proximal hub 112 may include internal threads configured to rotatably and/or threadably engage an externally threaded distal end of the core wire 30. Other configurations for releasably securing the left atrial appendage closure device 100 to the core wire 30 are also contemplated. As noted herein, some features are not shown in every figure to improve clarity.

The expandable framework 110 and/or the plurality of struts may be formed and/or cut from a tubular member. In some embodiments, the expandable framework 110 and/or the plurality of struts may be integrally formed and/or cut from a unitary member. In some embodiments, the expandable framework 110 and/or the plurality of struts may be integrally formed and/or cut from a unitary tubular member and subsequently formed and/or heat set to a desired shape in the fully unconstrained configuration. In some embodiments, the expandable framework 110 and/or the plurality of struts may be integrally formed and/or cut from a unitary flat member or sheet, and then rolled or formed into a tubular structure and subsequently formed and/or heat set to the desired shape in the fully unconstrained configuration. Some exemplary means and/or methods of making and/or forming the expandable framework 110 and/or the plurality of struts include laser cutting, machining, punching, stamping, electro discharge machining (EDM), chemical dissolution, etc. Other means and/or methods are also contemplated.

As would be understood by the skilled person, anatomical features may vary in size and/or shape. In some embodiments, the left atrial appendage may have an irregular (e.g., elongated and/or oblong) cross-sectional shape. In some embodiments, the expandable framework 110 may be compliant and substantially conform to and/or be in sealing engagement with the shape and/or geometry of a lateral wall of a left atrial appendage when deployed and/or expanded therein. In some embodiments, the left atrial appendage closure device 100 may expand to a size, extent, or shape less than or different from the fully unconstrained configuration, as determined by the surrounding tissue and/or lateral wall of the left atrial appendage. In some embodiments, the expandable framework 110 may be configured to shape and/or stretch the tissue of the left atrial appendage such that the lateral wall of the left atrial appendage substantially conforms to an outer shape of the expandable framework 110. Other configurations are also contemplated.

In some embodiments, the expandable framework 110 may include at least one anchoring member 116 extending radially outward therefrom in the fully unconstrained configuration. In some embodiments, the expandable framework 110 may include at least one anchoring member 116 extending radially outward from the expandable framework 110. In some embodiments, the expandable framework 110 may include at least one anchoring member 116 extending radially outward from the expandable framework 110 proximate a proximal shoulder of the expandable framework 110. In some embodiments, the expandable framework 110 may include at least one anchoring member 116 extending radially outward from the expandable framework 110 proximate a midsection of the expandable framework 110. In some embodiments, the at least one anchoring member 116 may be configured to engage with the lateral wall of the main body of the left atrial appendage. In some embodiments, the at least one anchoring member 116 may be formed as J-shaped hooks having a free end extending in and/or directed toward a proximal direction with respect to the central longitudinal axis of the left atrial appendage closure device 100 and/or the expandable framework 110. Other configurations are also contemplated.

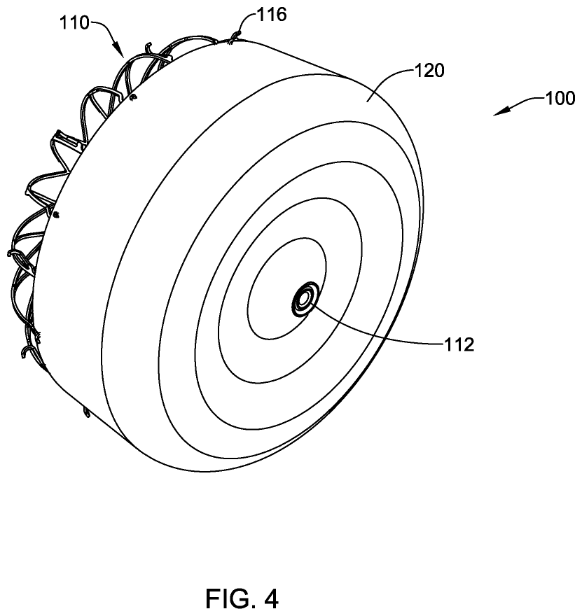

In some embodiments, the left atrial appendage closure device 100 may optionally include the occlusive element 120 connected to, disposed on, disposed over, disposed about, and/or disposed radially outward of at least a portion of the expandable framework 110 and/or the plurality of struts, as seen in FIG. 4. In some embodiments, the occlusive element 120 may be attached to the proximal hub 112 and/or may be attached to the expandable framework at the proximal hub 112. In some embodiments, the occlusive element 120 may extend radially outward from and/or may extend distally from the proximal hub 112. In some embodiments, the occlusive element 120 may be attached and/or secured to the expandable framework 110 at a plurality of discrete locations. In some embodiments, one of, some of, and/or all of the at least one anchoring member 116 may extend through an occlusive element 120, where present.

In some embodiments, the occlusive element 120 may include a membrane, a fabric, a mesh, a tissue element, or another suitable construction. In some embodiments, the occlusive element 120 may be porous. In some embodiments, the occlusive element 120 may be non-porous. In some embodiments, the occlusive element 120 may be permeable to selected gases and/or fluids. In some embodiments, the occlusive element 120 may be substantially impermeable to selected gases and/or fluids, such as blood, water, etc. In some embodiments, the occlusive element 120 may be designed, sized, and/or configured to prevent thrombus and/or embolic material from passing out of the left atrial appendage into the left atrium and/or the patient's bloodstream. In some embodiments, the occlusive element 120 may be configured to promote endothelization after implantation, thereby effectively removing the target site (e.g., the left atrial appendage, etc.) from the patient's circulatory system. Some suitable, but non-limiting, examples of materials for the occlusive element 120 are discussed below.

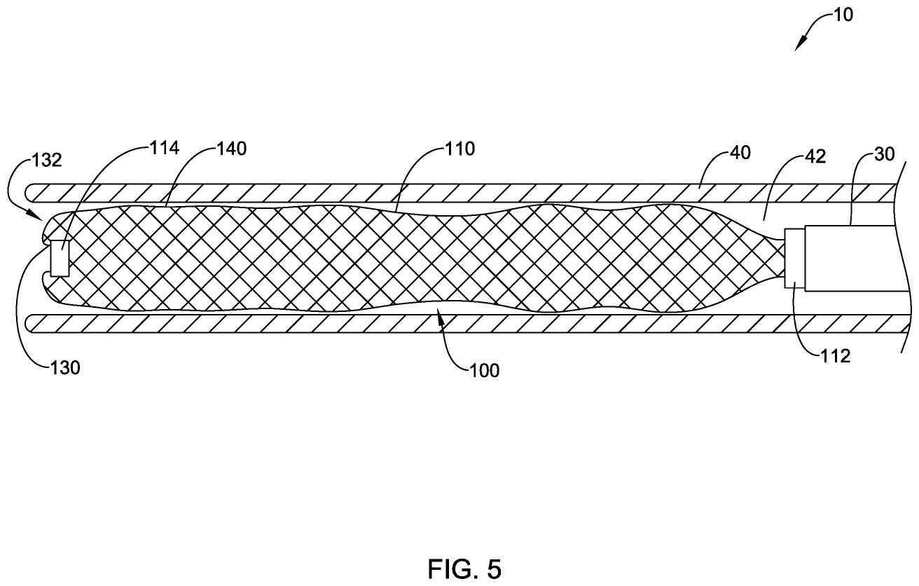

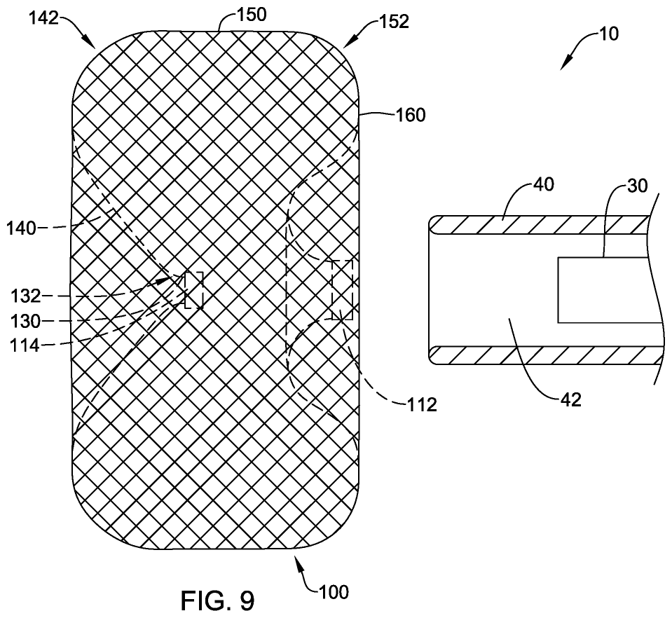

FIGS. 5-9 schematically illustrate selected aspects of the left atrial appendage closure device 100 and/or the left atrial appendage closure device system 10 during deployment of the left atrial appendage closure device 100. For clarity and ease of understanding, some elements of the left atrial appendage closure device 100 are not shown but shall be understood to be present in accordance and/or consistent with other figures and/or description of the disclosure. FIGS. 5-8 illustrate the left atrial appendage closure device 100 in partial cross-section. FIG. 9 illustrates the left atrial appendage closure device 100 using broken lines to show hidden features as may be understood from other figures. The occlusive element 120 is not shown in FIG. 9. It may be seen in FIGS. 5-9 that as the expandable framework 110 of the left atrial appendage closure device 100 shifts from fully constrained to fully unconstrained, the expandable framework 110 may transition sequentially through a plurality of positions. In some embodiments, the plurality of positions may include a first position, a second position, a third position, a fourth position, and/or a fifth position as described herein. In some embodiments, the plurality of positions may include additional and/or other positions.

Returning now to FIG. 5, in some embodiments, the expandable framework 110 may be fully constrained in a first position by the delivery sheath 40. In some embodiments, the expandable framework 110 may be disposed within the lumen 42 of the delivery sheath 40 in the first position. In some embodiments, in the first position a first segment 130 of the plurality of struts of the expandable framework 110 may extend distally from the distal hub 114 substantially parallel to the central longitudinal axis to a first bend 132 and a second segment 140 of the plurality of struts of the expandable framework 110 may extend from the first bend 132 proximally. In some embodiments, in the first position the second segment 140 may extend from the first bend 132 proximally and generally parallel to the central longitudinal axis. In the first position, the distal hub 114 may be disposed proximal of the first bend 132. In some embodiments, the first segment 130 may be secured to the distal hub 114. In some embodiments, the first segment 130 may be fixedly attached to the distal hub 114. For example, the first segment 130 may be welded, adhesively bonded, etc. to the distal hub 114. Other configurations are also contemplated.

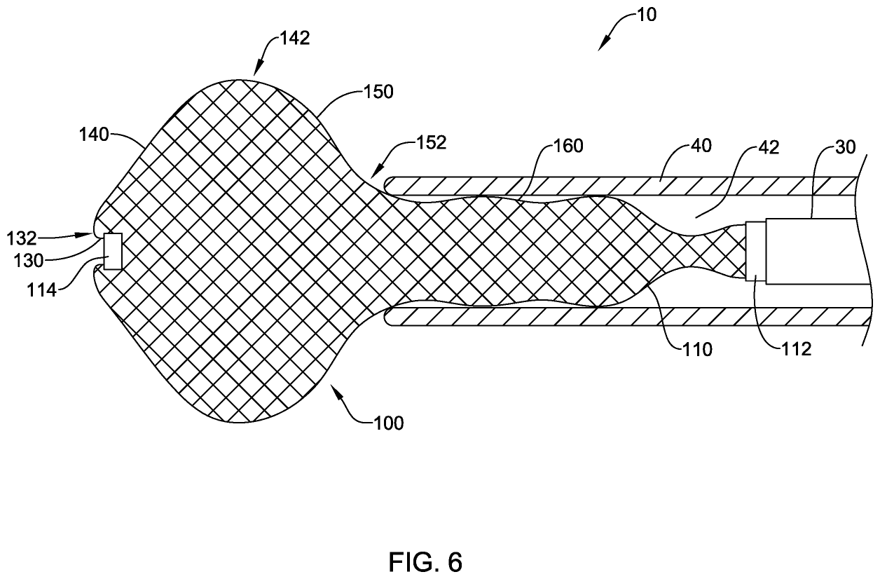

In some embodiments, relative axial translation between the delivery sheath 40 and the expandable framework 110 may expose some of the expandable framework 110 and/or the plurality of struts in a second position, as seen in FIG. 6. In some embodiments, a first amount of the expandable framework 110 and/or the plurality of struts may be exposed from and/or unconstrained by the delivery sheath 40 in the second position. In some embodiments, the first amount may be less than about 55% of an axial length, a volume, a weight, and/or a surface area of the expandable framework 110 and/or the plurality of struts. In some embodiments, the first amount may be about 15% to about 55% of the axial length, the volume, the weight, and/or the surface area of the expandable framework 110 and/or the plurality of struts. Other configurations and/or ranges are also contemplated. In some embodiments, in the second position, the expandable framework 110 may have a maximum radial extent of about 6 millimeters (mm) to about 10 mm. In some embodiments, in the second position, the expandable framework 110 may have a maximum radial extent of about 8 mm. In some embodiments, in the second position, the maximum radial extent of the expandable framework 110 may be about twice or about 200% of a maximum outer extent of a distal end of the delivery sheath 40. Other configurations and/or sizes are also contemplated.

In some embodiments, in the second position the first segment 130 of the plurality of struts of the expandable framework 110 may extend distally from the distal hub 114 substantially parallel to the central longitudinal axis to the first bend 132. In the second position, the second segment 140 of the plurality of struts of the expandable framework 110 may extend from the first bend 132 proximally and radially outward toward and/or to a second bend 142. In the second position, a third segment 150 of the plurality of struts of the expandable framework 110 may extend from the second bend 142 proximally and radially inward toward and/or to a third bend 152. In some embodiments, in the second position, a fourth segment 160 of the plurality of struts of the expandable framework 110 may extend from the third bend 152 proximally to within the lumen 42 of the delivery sheath 40. In some embodiments, in the second position, the fourth segment 160 of the plurality of struts of the expandable framework 110 may extend from the third bend 152 proximally toward the proximal hub 112 disposed within the lumen 42 of the delivery sheath 40.

In some embodiments, in the second position, the distal hub 114 is disposed proximal of the first bend 132. In some embodiments, in the second position, the distal hub 114 may be disposed distal of the second bend 142. For example, in the second position, the distal hub 114 may be positioned distal of a plane extending through, including at least a portion of, and/or tangent to the second bend 142 and oriented perpendicular to the central longitudinal axis. In some embodiments, in the second position, the first segment 130 of the plurality of struts and the second segment 140 of the plurality of struts may form and/or intersect to form an acute angle opening inwardly toward an interior of the expandable framework 110. In some embodiments, in the second position, the first segment 130 of the plurality of struts and the second segment 140 of the plurality of struts may form and/or intersect to form an acute angle opening radially outwardly from the central longitudinal axis. In some embodiments, in the second position, the second segment 140 of the plurality of struts of the expandable framework 110, if swept circumferentially around the central longitudinal axis, may define a generally conical shape tapering radially outward in a proximal direction from the first bend 132 toward the second bend 142.

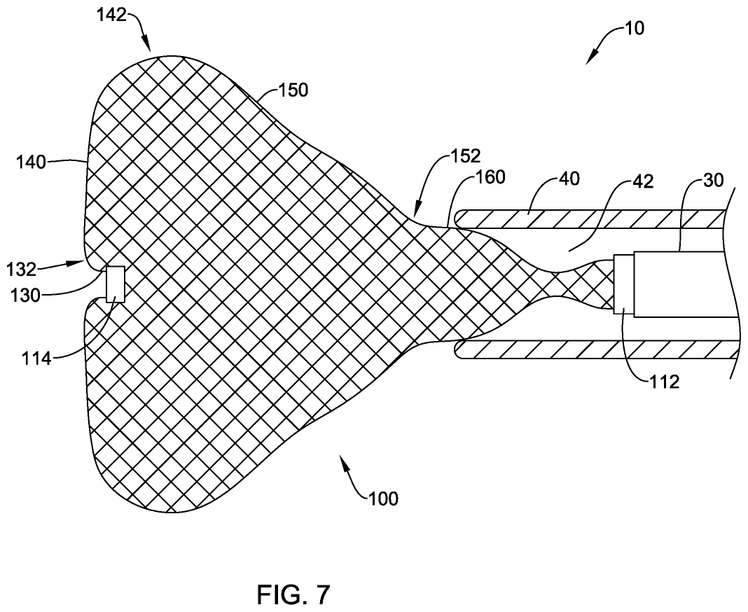

In some embodiments, relative axial translation between the delivery sheath 40 and the expandable framework 110 may expose more of the expandable framework 110 and/or the plurality of struts in a third position than in the second position, as seen in FIG. 7. In some embodiments, a second amount of the expandable framework 110 and/or the plurality of struts greater than the first amount may be exposed from and/or unconstrained by the delivery sheath 40 in the third position. In some embodiments, the second amount may be less than about 75% of the axial length, the volume, the weight, and/or the surface area of the expandable framework 110 and/or the plurality of struts. In some embodiments, the second amount may be about 40% to about 75% of the axial length, the volume, the weight, and/or the surface area of the expandable framework 110 and/or the plurality of struts. Other configurations and/or ranges are also contemplated.

In some embodiments, in the third position the first segment 130 of the plurality of struts of the expandable framework 110 may extend distally from the distal hub 114 substantially parallel to the central longitudinal axis to the first bend 132. In the third position, the second segment 140 of the plurality of struts of the expandable framework 110 may extend from the first bend 132 radially outward generally perpendicular to the central longitudinal axis toward and/or to the second bend 142. In the third position, the third segment 150 of the plurality of struts of the expandable framework 110 may extend from the second bend 142 proximally and radially inward toward and/or to the third bend 152. In some embodiments, in the third position, the fourth segment 160 of the plurality of struts of the expandable framework 110 may extend from the third bend 152 proximally to within the lumen 42 of the delivery sheath 40. In some embodiments, in the third position, the fourth segment 160 of the plurality of struts of the expandable framework 110 may extend from the third bend 152 proximally toward the proximal hub 112 disposed within the lumen 42 of the delivery sheath 40.

In some embodiments, in the third position, the distal hub 114 is disposed proximal of the first bend 132. In some embodiments, in the third position, the first segment 130 of the plurality of struts and the second segment 140 of the plurality of struts may form and/or intersect to form a generally right angle opening toward the interior of the expandable framework 110. In some embodiments, in the third position, the first segment 130 of the plurality of struts and the second segment 140 of the plurality of struts may form and/or intersect to form a generally right angle opening radially outwardly away from the central longitudinal axis. In some embodiments, in the third position, the second segment 140 of the plurality of struts and the third segment 150 of the plurality of struts may form and/or intersect to form an acute angle opening radially inwardly toward the interior of the expandable framework 110. In some embodiments, in the third position, the second segment 140 of the plurality of struts and the third segment 150 of the plurality of struts may form and/or intersect to form an acute angle opening radially inwardly toward the central longitudinal axis. In some embodiments, in the third position, the third segment 150 of the plurality of struts and the fourth segment 160 of the plurality of struts may form and/or intersect to form an obtuse angle opening radially outwardly away from the central longitudinal axis. In some embodiments, in the third position, the second segment 140 of the plurality of struts of the expandable framework 110, if swept circumferentially around the central longitudinal axis, may define a generally planar shape oriented generally perpendicular to the central longitudinal axis.

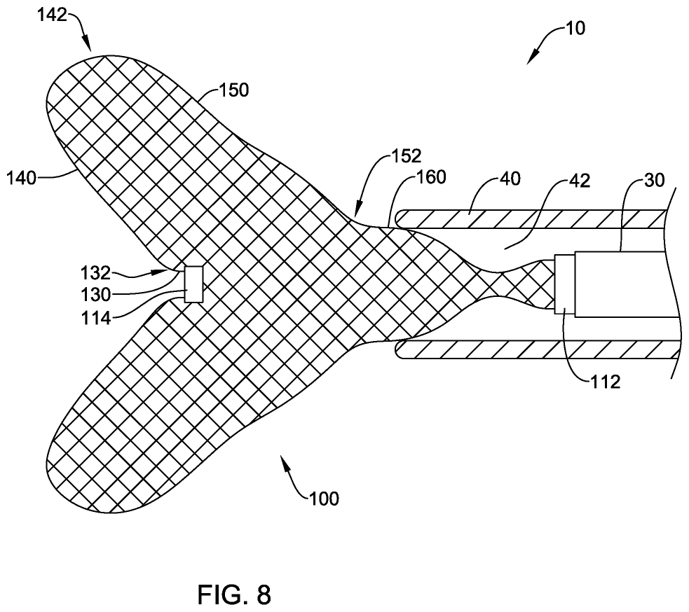

In some embodiments, relative axial translation between the delivery sheath 40 and the expandable framework 110 may expose more of the expandable framework 110 and/or the plurality of struts in a fourth position than in the third position, as seen in FIG. 8. In some embodiments, a third amount of the expandable framework 110 and/or the plurality of struts greater than the second amount may be exposed from and/or unconstrained by the delivery sheath 40 in the fourth position. In some embodiments, the third amount may be less than about 95% of the axial length, the volume, the weight, and/or the surface area of the expandable framework 110 and/or the plurality of struts. In some embodiments, the third amount may be about 60% to about 95% of the axial length, the volume, the weight, and/or the surface area of the expandable framework 110 and/or the plurality of struts. Other configurations and/or ranges are also contemplated.

In some embodiments, in the fourth position the first segment 130 of the plurality of struts of the expandable framework 110 may extend distally from the distal hub 114 substantially parallel to the central longitudinal axis to the first bend 132. In the fourth position, the second segment 140 of the plurality of struts of the expandable framework 110 may extend from the first bend 132 distally and radially outward toward and/or to the second bend 142. In the fourth position, the third segment 150 of the plurality of struts of the expandable framework 110 may extend from the second bend 142 proximally and radially inward toward and/or to the third bend 152. In some embodiments, in the fourth position, the fourth segment 160 of the plurality of struts of the expandable framework 110 may extend from the third bend 152 proximally toward the proximal hub 112 disposed within the lumen 42 of the delivery sheath 40.

In some embodiments, in the fourth position, the distal hub 114 is disposed proximal of the first bend 132. In some embodiments, in the fourth position, the first segment 130 of the plurality of struts and the second segment 140 of the plurality of struts may form and/or intersect to form an obtuse angle opening inwardly toward the interior of the expandable framework 110. In some embodiments, in the fourth position, the second segment 140 of the plurality of struts and the third segment 150 of the plurality of struts may form and/or intersect to form an acute angle opening radially inwardly toward the central longitudinal axis. In some embodiments, in the fourth position, the third segment 150 of the plurality of struts and the fourth segment 160 of the plurality of struts may form and/or intersect to form an obtuse angle opening radially outwardly away from the central longitudinal axis. In some embodiments, in the fourth position, the second segment 140 of the plurality of struts of the expandable framework 110, if swept circumferentially around the central longitudinal axis, may define a generally conical shape tapering radially outward in a distal direction from the first bend 132 toward the second bend 142.

In some embodiments, relative axial translation between the delivery sheath 40 and the expandable framework 110 may expose substantially all of the expandable framework 110 and/or the plurality of struts in a fifth position, as seen in FIG. 9. In some embodiments, a fourth amount of the expandable framework 110 and/or the plurality of struts greater than the third amount may be exposed from and/or unconstrained by the delivery sheath 40 in the fifth position. In some embodiments, the fourth amount may be more than about 95% of the axial length, the volume, the weight, and/or the surface area of the expandable framework 110 and/or the plurality of struts. In some embodiments, the fourth amount may be about 100% of the axial length, the volume, the weight, and/or the surface area of the expandable framework 110 and/or the plurality of struts. Other configurations and/or ranges are also contemplated.

In some embodiments, the expandable framework 110 may be in the fully unconstrained configuration in the fifth position. In some embodiments, in the fully unconstrained configuration, the expandable framework may have a maximum radial extent of about 16 millimeters (mm) to about 40 mm. In some embodiments, in the fully unconstrained configuration, the expandable framework may have a maximum radial extent of about 16 mm. In some embodiments, in the fully unconstrained configuration, the expandable framework may have a maximum radial extent of about 20 mm. In some embodiments, in the fully unconstrained configuration, the expandable framework may have a maximum radial extent of about 25 mm. In some embodiments, in the fully unconstrained configuration, the expandable framework may have a maximum radial extent of about 30 mm. In some embodiments, in the fully unconstrained configuration, the expandable framework may have a maximum radial extent of about 35 mm. In some embodiments, in the fully unconstrained configuration, the expandable framework may have a maximum radial extent of about 40 mm. Other configurations and/or sizes are also contemplated.

In some embodiments, in the fifth position the first segment 130 of the plurality of struts of the expandable framework 110 may extend distally from the distal hub 114 substantially parallel to the central longitudinal axis to the first bend 132. In the fifth position, the second segment 140 of the plurality of struts of the expandable framework 110 may extend from the first bend 132 distally and radially outward toward and/or to the second bend 142. In the fifth position, the third segment 150 of the plurality of struts of the expandable framework 110 may extend from the second bend 142 proximally and toward and/or to the third bend 152. In some embodiments, the fifth position, the third segment 150 of the plurality of struts of the expandable framework 110 may extend from the second bend 142 proximally generally parallel to the central longitudinal axis toward and/or to the third bend 152. In some embodiments, in the fifth position, the fourth segment 160 of the plurality of struts of the expandable framework 110 may extend from the third bend 152 radially inward toward and/or to the proximal hub 112.

In some embodiments, in the fifth position, the distal hub 114 is disposed proximal of the first bend 132. In some embodiments, in the fifth position, the first segment 130 of the plurality of struts and the second segment 140 of the plurality of struts may form and/or intersect to form an obtuse angle opening inwardly toward the interior of the expandable framework 110. In some embodiments, in the fifth position, the second segment 140 of the plurality of struts and the third segment 150 of the plurality of struts may form and/or intersect to form an acute angle opening radially inwardly toward the central longitudinal axis. In some embodiments, in the fifth position, the third segment 150 of the plurality of struts and the fourth segment 160 of the plurality of struts may form and/or intersect to form an angle of about 90 degrees or less opening radially inwardly toward the interior of the expandable framework 110. In some embodiments, in the fifth position, the third segment 150 of the plurality of struts and the fourth segment 160 of the plurality of struts may form and/or intersect to form an angle of about 90 degrees or less opening radially inwardly toward the central longitudinal axis.

A method for occluding the left atrial appendage may comprise advancing the left atrial appendage closure device 100 into the left atrial appendage of the patient's heart. For example, the left atrial appendage closure device 100 may be advanced to the left atrial appendage within the lumen 42 of the delivery sheath 40 in the fully constrained configuration. The method may include deploying the expandable framework 110 from the delivery sheath 40 within the left atrial appendage. The method may further include expanding and/or shifting the expandable framework 110 from the fully constrained configuration toward the fully unconstrained configuration within the left atrial appendage.

As the expandable framework 110 shifts from fully constrained to fully unconstrained, the expandable framework 110 may transition sequentially through a plurality of positions, as described herein. In some embodiments, in the second position, the expandable framework 110 may be moved and/or navigated within the patient's heart, the left atrium, and/or the left atrial appendage. In the second position, the expandable framework 110 may form a generally rounded atraumatic shape. In some embodiments, in the second position, the maximum radial extent of the expandable framework 110 may be about twice or about 200% of a maximum outer extent of a distal end of the delivery sheath 40. In some embodiments, in the second position, the maximum radial extent of the expandable framework 110 may be about 8 millimeters. Other configurations and/or sizes are also contemplated. In some embodiments, a physician may use the generally rounded atraumatic shape as a navigational tool within the patient's anatomy.

In and/or near the fully unconstrained configuration (e.g., the fifth position), the expandable framework 110 may be urged into contact with, engaged with, and/or anchored to the lateral wall of the main body of the left atrial appendage. In some embodiments, the expandable framework 110 may not fully achieve the fully unconstrained configuration (e.g., the fifth position) due to contact with the wall(s) of the left atrial appendage. However, the inverted shape of the distal portion of the expandable framework 110 (e.g., the first segment 130, the first bend 132, the second segment 140, and the second bend 142) may prevent the expandable framework 110 from elongating distally, which may compromise locating the left atrial appendage closure device 100 and/or the expandable framework 110 within the left atrial appendage, sealing of the left atrial appendage closure device 100 and/or the expandable framework 110 with respect to the left atrial appendage, and/or anchoring of the left atrial appendage closure device 100 and/or the expandable framework 110 within the left atrial appendage. Instead, compressive force applied against the third segment 150 and/or the second bend 142 may urge the distal hub 114 proximally toward the proximal hub 112. As such, the final shape of the left atrial appendage closure device 100 and/or the expandable framework 110 may be more predictable and locating and anchoring of the left atrial appendage closure device 100 and/or the expandable framework 110 within the left atrial appendage may be improved.

In at least some embodiments, the left atrial appendage closure device 100 and/or the expandable framework 110 may span across the ostium of the left atrial appendage. In some embodiments, the left atrial appendage closure device 100 and/or the expandable framework 110 may span completely across the ostium of the left atrial appendage, thereby effectively removing the left atrial appendage from the circulatory system of the patient.

When satisfied with the positioning of the left atrial appendage closure device 100 within the left atrial appendage, the core wire 30 may be disconnected from the left atrial appendage closure device 100, thereby leaving the left atrial appendage closure device 100 disposed at and/or in the left atrial appendage. In some embodiments, disconnecting the core wire 30 from the left atrial appendage closure device 100 may include rotating the externally threaded distal end of the core wire 30 relative to the left atrial appendage closure device 100 and/or the proximal hub 112 to disengage the core wire 30 from the left atrial appendage closure device 100.

In some embodiments, the delivery sheath 40 and/or the core wire 30 may include a keying structure configured to prevent rotation of the core wire 30 relative to the proximal hub 112. In such embodiments, the keying structure is disengaged prior to rotating the core wire 30 relative to the left atrial appendage closure device 100 and/or the proximal hub 112. When the keying structure is engaged, rotation of the core wire 30 may be transmitted to left atrial appendage closure device 100 and/or the expandable framework 110. In some embodiments, rotation of the left atrial appendage closure device 100 and/or the expandable framework 110 may facilitate positioning and/or orientation of the left atrial appendage closure device 100 and/or the expandable framework 110 relative to the left atrial appendage, for example, with respect to an asymmetrical and/or irregular ostium and/or left atrial appendage. Other configurations, purposes, and/or results are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the left atrial appendage closure device, the delivery sheath, the core wire, the expandable framework, the occlusive element, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the system and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or other elements disclosed herein to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A left atrial appendage closure device, comprising:
an expandable framework configured to shift between a constrained configuration and an expanded configuration, the expandable framework having a plurality of struts disposed about a central longitudinal axis, the plurality of struts being joined together at a proximal hub and a distal hub;
wherein, in the expanded configuration:
a distal portion of the expandable framework includes a first segment of the plurality of struts extending distally from the distal hub to a first bend, and a second segment of the plurality of struts extending from the first bend distally and radially outward to a second bend; and
the distal hub is disposed proximal of the first bend.

2. The device of claim 1, wherein the second segment, if swept circumferentially around the central longitudinal axis, defines a generally conical shape tapering radially outward in a distal direction from the first bend toward the second bend.

3. The device of claim 1, wherein, in the expanded configuration, a third segment of the plurality of struts extends proximally from the second bend toward a third bend.

4. The device of claim 3, wherein the third segment extends generally parallel to the central longitudinal axis.

5. The device of claim 3, wherein a fourth segment of the plurality of struts extends from the third bend radially inward toward the proximal hub.

6. The device of claim 5, wherein the third segment and the fourth segment form an angle of about 90 degrees or less opening inwardly.

7. The device of claim 3, wherein the second segment and the third segment form an acute angle opening toward the central longitudinal axis.

8. The device of claim 1, wherein the first segment extends substantially parallel to the central longitudinal axis.

9. The device of claim 1, wherein the first segment and the second segment form an obtuse angle opening inwardly toward an interior of the expandable framework.

10. The device of claim 1, wherein the expandable framework is self-biased toward the expanded configuration.

11. The device of claim 1, wherein the plurality of struts are integrally formed from a unitary tubular member.

12. The device of claim 1, further comprising an occlusive element disposed radially outward of at least a portion of the expandable framework.

13. The device of claim 12, wherein the occlusive element is attached to the proximal hub.

14. The device of claim 12, further comprising at least one anchoring member extending radially outward from the expandable framework.

15. The device of claim 14, wherein the at least one anchoring member extends radially outward from the expandable framework and through the occlusive element.

16. A left atrial appendage closure device, comprising:
an expandable framework having a plurality of struts disposed about a central longitudinal axis, the plurality of struts being joined together at a proximal hub and a distal hub;
wherein the expandable framework is configured to shift from a constrained configuration toward an expanded configuration through a plurality of configurations;
wherein, in a first configuration, a first segment of the plurality of struts extends distally from the distal hub to a first bend and a second segment of the plurality of struts extends distally and radially outward from the first bend;
wherein, in a second configuration, the second segment of the plurality of struts, if swept circumferentially around the central longitudinal axis, defines a generally conical shape tapering radially outward in a distal direction from the first bend toward a second bend.

17. The device of claim 16, wherein, in the second configuration, the first segment of the plurality of struts and the second segment of the plurality of struts form an obtuse angle opening inwardly toward the interior of the expandable framework.

18. The device of claim 16, wherein, in the second configuration, the distal hub is disposed proximally of the first bend.

19. The device of claim 16, wherein, in a third configuration, a third segment of the plurality of struts extends proximally from the second bend toward a third bend.

20. A left atrial appendage closure device, comprising:
an expandable framework configured to shift between a constrained configuration and an expanded configuration, the expandable framework having a plurality of struts disposed about a central longitudinal axis, the plurality of struts being joined together at a proximal hub and a distal hub;
wherein, in the expanded configuration:
a distal portion of the expandable framework includes a first segment of the plurality of struts extending distally from the distal hub to a first bend, and a second segment of the plurality of struts extending from the first bend to a second bend;
the distal hub is disposed proximal of the first bend; and
the first segment and the second segment form an obtuse angle opening inwardly toward an interior of the expandable framework.
